(19) United States
(12) Patent Application Publication
(10) Pub. No.: US 2024/0099724 A1
(43) Pub. Date: Mar. 28, 2024

(54) TWO-PART FASTENERS FOR SURGICAL CLIP APPLIERS AND SURGICAL CLIP APPLIERS FOR DEPLOYING THE SAME

(71) Applicant: Covidien LP, Mansfield, MA (US)

(72) Inventors: Jacob C. Baril, Norwalk, CT (US); Saumya Banerjee, Southington, CT (US); Garrett P. Ebersole, Hamden, CT (US)

(21) Appl. No.: 17/952,776

(22) Filed: Sep. 26, 2022

(51) Int. Cl. A61B 17/128

(52) U.S. Cl. CPC A61B 17/1285; A61B 2017/00407

(57) ABSTRACT

A surgical clip applier includes an elongated portion, a drive shaft, and an end effector. The drive shaft extends at least partially through the elongated portion. The end effector is disposed adjacent a distal end of the elongated portion, and includes a collar and a distal housing. The collar is coupled to the drive shaft and is movable relative to the distal housing. The collar is configured to support a base of a two-part fastener. The distal housing includes a leg and a C-shaped portion extending from a distal end of the leg. The C-shaped portion is configured to support a clip of the two-part fastener.

### BACKGROUND

This disclosure relates to surgical instruments and to fasteners for use with surgical instruments. More particularly, this disclosure relates to two-part surgical clips and surgical clip appliers for deploying the same.

Surgical clip appliers are known in the art and are used for a number of distinct and useful surgical procedures including endoscopic, open, and robotic surgical procedures. Surgical clip appliers having various sizes (e.g., diameters) are configured to apply a variety of diverse surgical clips, and are capable of applying a single or multiple surgical clips within the body cavity. Such surgical clips are typically fabricated from a biocompatible material and are usually compressed over tissue. Once applied to tissue, the compressed surgical clip terminates the flow of fluid through the tissue.

Certain surgical procedures or situations may benefit from the implementation of a partially-formed surgical clip to crimp a vessel, or to retain a catheter in a vessel without occluding the vessel (e.g., during cholangiograms), for instance.

### SUMMARY

This disclosure relates to a surgical clip applier including an elongated portion, a drive shaft, and an end effector. The elongated portion defines a longitudinal axis and includes a distal end. The drive shaft extends at least partially through the elongated portion. The end effector is disposed adjacent the distal end of the elongated portion, and includes a collar and a distal housing. The collar is coupled to the drive shaft and is movable relative to the distal housing. The collar is configured to support a base of a two-part fastener. The distal housing includes a leg and a C-shaped portion extending from a distal end of the leg. The C-shaped portion of the distal housing is configured to support a clip of the two-part fastener.

In disclosed embodiments, a distal-most end of the C-shaped portion of the distal housing defines an aperture. In embodiments, the C-shaped portion of the distal housing defines a slot extending laterally from the aperture. The slot is configured to support a portion of the clip of the two-part fastener.

In disclosed embodiments, the distal housing is fixed from longitudinal movement relative to the elongated portion.

In disclosed embodiments, the collar of the end effector defines a recess for accepting the base of the two-part fastener partially in the recess.

In disclosed embodiments, the collar of the end effector defines a first leg aperture and a second leg aperture. The first leg aperture is configured to allow a first leg of the clip of the two-part fastener to extend through the collar. The second leg aperture is configured to allow a second leg of the clip of the two-part fastener to extend through the collar. In embodiments, the first leg aperture and the second leg aperture are defined within a recess of the collar.

This disclosure also relates to a surgical clip-applying system including a surgical clip applier and a two-part fastener. The surgical clip applier includes an elongated portion defining a longitudinal axis and including a distal end. The end effector is disposed adjacent the distal end of the elongated portion, and includes a collar and a distal housing. At least one of the collar or the distal housing is movable along the longitudinal axis relative to the elongated portion. The two-part fastener includes a base and a clip. The base is configured to be supported by the collar of the end effector of the surgical clip applier. The clip is configured to be supported by the distal housing of the end effector of the surgical clip applier, and includes a first leg and a second leg. A predetermined amount movement of at least one of the collar or the distal housing relative to the elongated portion causes the base of the two-part fastener to engage the clip of the two-part fastener.

In disclosed embodiments, the distal housing of the end effector includes a C-shaped portion.

In disclosed embodiments, the base of the two-part fastener defines a first aperture and a second aperture. In embodiments, the clip of the two-part fastener is C-shaped and includes a first leg and a second leg. The first leg is configured to selectively extend through the first aperture of the base of the two-part fastener, and the second leg configured to selectively extend through the second aperture of the base of the two-part fastener. In embodiments, each of the first leg and the second leg of the clip of the two-part fastener includes a plurality of teeth configured to engage the base of the two-part fastener in a ratcheting manner.

In disclosed embodiments, a distal-most end of the distal housing of the end effector defines an aperture. In embodiments, a distal end of the clip of the two-part fastener includes a distal tip configured to selectively extend through the aperture of the distal housing of the end effector. In embodiments, the distal tip of the clip of the two-part fastener defines a slit extending generally parallel to the first leg of the clip of the two-part fastener. In embodiments, when the distal tip of the clip of the two-part fastener is extended through the aperture of the distal housing of the end effector, the slit of the distal tip extends distally beyond the distal-most end of the distal housing of the end effector.

In disclosed embodiments, the base of the two-part fastener is supported partially within a recess of the collar of the end effector.

In disclosed embodiments, the collar of the end effector defines a first aperture and a second aperture, and the base of the two-part fastener defines a first aperture and a second aperture. In embodiments, after a predetermined amount of movement of the at least one of the collar or the distal housing relative to the elongated portion, the first leg of the clip of the two-part fastener extends through the first aperture of the collar and through the first aperture of the base of the two-part fastener, and the second leg of the clip of the two-part fastener extends through the second aperture of the collar and through the second aperture of the base of the two-part fastener.

This disclosure also relates to a two-part fastener for use with a surgical clip applier. The two-part fastener includes a base and a clip. The base includes a rectangular portion, a first aperture extending through the rectangular portion, and a second aperture extending through the rectangular portion. The clip includes a first leg, a second leg, and a distal tip interconnecting the first leg and the second leg. The first leg includes a plurality of teeth and is configured to selectively extend at least partially through the first aperture of the base. The second leg includes a plurality of teeth and is configured to selectively extend at least partially through the second aperture of the base.

In disclosed embodiments, the first leg of the clip is generally parallel to the second leg of the clip.

In disclosed embodiments, at least a portion of the distal tip is generally parallel to the first leg and the second leg. In embodiments, the distal tip defines a slit extending through a proximal portion of the distal tip.

In disclosed embodiments, the first leg of the base is configured to selectively extend completely through the first aperture of the base. In embodiments, the second leg of the base is configured to selectively extend completely through the second aperture of the base.

In disclosed embodiments, the base is configured to engage the clip in a plurality of discrete positions.

This disclosure additionally relates to a method of fixing a catheter to a vessel. The method includes positioning an end effector of a surgical clip applier adjacent the vessel such that a distal housing of the end effector is positioned distally of the vessel, and a collar of the end effector is positioned proximally of the vessel. The method also includes moving the collar of the end effector distally such that a base of a two-part fastener supported by the collar moves toward a clip of the two-part fastener supported by the distal housing of the end effector. The method also includes moving the collar of the end effector distally such that a first aperture of the base of the two-part fastener engages a first leg of the clip of the two-part fastener and a second aperture of the base engages a second leg of the clip. The method also includes moving the collar of the end effector distally such that the base of the two-part fastener is in a desired discrete position relative to the clip of the two-part fastener such that the catheter is sufficiently fixed to the vessel and such that the vessel is not occluded.

In disclosed embodiments, moving the collar of the end effector distally until the base of the two-part fastener is in a desired discrete position relative to the clip of the two-part fastener includes moving the collar from a first discrete position to a second discrete position.

In disclosed embodiments, the method also includes moving the collar of the end effector distally such that the first leg of the clip of the two-part fastener extends through a first aperture of the collar, and such that the second leg of the clip of the two-part fastener extends through a second aperture of the collar.

In disclosed embodiments, the method also includes moving the collar proximally relative to the base of the two-part fastener. In embodiments, the method also includes moving the distal housing of the end effector distally relative to the clip of the two-part fastener such that a distal tip of the clip moves through an aperture of the distal housing. In embodiments, moving the distal housing of the end effector distally relative to the clip of the two-part fastener is performed after moving the collar proximally relative to the base of the two-part fastener. In embodiments, the method also includes cutting the distal tip of the clip of the two-part fastener to separate the first leg from the second leg of the clip of the two-part fastener.

### BRIEF DESCRIPTION OF THE DRAWINGS

Embodiments of the disclosure are described in detail with reference to the drawing figures wherein like reference numerals identify similar or identical structural elements and:

FIG. 1 is a front, perspective view of an endoscopic surgical clip applier illustrating an end effector in an open position and supporting a two-part fastener, in accordance with embodiments of the disclosure;

FIG. 2 is a side view of a handle assembly of the surgical clip applier of FIG. 1, with portions removed, and illustrating a trigger of the surgical clip applier disposed in an un-actuated position;

FIG. 3 is a side view of the handle assembly of the surgical clip applier of FIG. 1, with portions removed, and illustrating the trigger in an actuated position;

FIG. 4 is an enlarged view of the area of detail indicated in FIG. 1 illustrating a distal end of the surgical clip applier supporting the two-part fastener;

FIG. 5 is a perspective, exploded view of the distal end of the surgical clip applier and two-part fastener of FIG. 1;

FIG. 6 is a perspective, exploded view of a portion of the two-part fastener and a portion of the end effector of the surgical clip applier of FIG. 1;

FIG. 7 is a perspective view of a portion of the two-part fastener engaged with a portion of the end effector of the surgical clip applier of FIG. 1;

FIGS. 8 and 9 are perspective views of the surgical clip applier of FIG. 1 illustrating the end effector in various positions relative to an elongated portion of the surgical clip applier;

FIG. 10 is a perspective view of the two-part fastener engaged with the end effector of the surgical clip applier of FIG. 1 illustrating tissue and a catheter positioned between the two parts of the two-part fastener;

FIG. 11 is a cross-sectional view of the two-part fastener, end effector of the surgical clip applier, tissue and a catheter taken along line 11-11 in FIG. 10;

FIG. 12 is a perspective view of a proximal portion of the surgical clip applier of FIG. 1 illustrating a pivotable handle in a partially-actuated position;

FIG. 13 is a perspective view of a distal portion of the surgical clip applier of FIG. 1 corresponding to the pivotable handle being in the partially-actuated position of FIG. 12;

FIG. 14 is a cross-sectional view of the two-part fastener and end effector of the surgical clip applier taken along line 14-14 in FIG. 13, and further illustrating tissue and the catheter positioned between the two parts of the two-part fastener;

FIG. 15 is a cross-sectional view of the two-part fastener, end effector of the surgical clip applier of FIG. 1, tissue and the catheter positioned between the two parts of the two-part fastener, and corresponding to the pivotable handle of the surgical clip applier being in a further-actuated position;

FIG. 16 is a cross-sectional view of the two-part fastener, end effector of the surgical clip applier of FIG. 1, tissue and the catheter secured between the two parts of the two-part fastener, following retraction of a collar of the surgical clip applier;

FIG. 17 is a perspective view of the two-part fastener of FIG. 1 illustrated in an engaged orientation;

FIG. 18 is a perspective view of the two-part fastener of FIG. 1 illustrated in an engaged orientation and surrounding tissue and the catheter;

FIG. 19 is a perspective view of the two-part fastener of FIG. 1 after the two-part fastener has been cut and released from tissue and the catheter; and

FIG. 20 is a schematic illustration of a robotic surgical system configured for use in accordance with the disclosure.

### DETAILED DESCRIPTION

Embodiments of the disclosed surgical clip applier and two-part fastener are described in detail with reference to the drawings, in which like reference numerals designate identical or corresponding elements in each of the several views. Non-limiting examples of surgical clip appliers according to the disclosure include manual, robotic, mechanical and/or electromechanical, and the like. As used herein the term “distal” refers to that portion of the two-part fastener, surgical clip applier, or component thereof, farther from the user, while the term “proximal” refers to that portion of the two-part fastener, surgical clip applier, or component thereof, closer to the user.

As will be described in greater detail below, the disclosure includes a two-part fastener, and a surgical clip applier for utilizing the two-part fastener to fix a catheter to a vessel. Related methods of use are also encompassed by this disclosure.

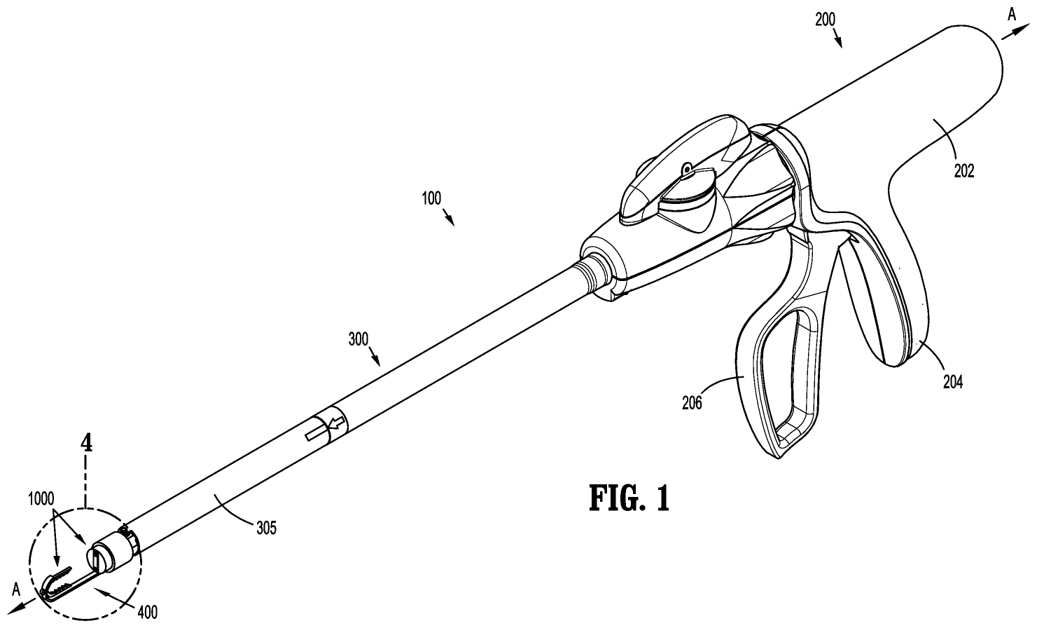

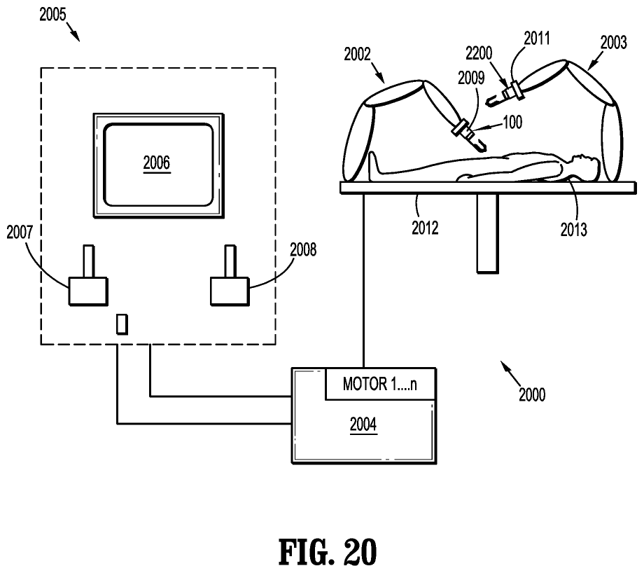

FIGS. 1-20 illustrate surgical clip appliers and two-part fasteners in accordance with embodiments of the disclosure. While the figures illustrate an endoscopic surgical clip applier (e.g., FIG. 1), and a robotic surgical clip applier (FIG. 20), other types of surgical clip appliers (e.g., open surgical clip appliers) are encompassed by the scope of the present disclosure and are usable with the disclosed two-part fasteners. For simplicity, each of the disclosed surgical clip appliers is generally referred to as surgical clip applier 100.

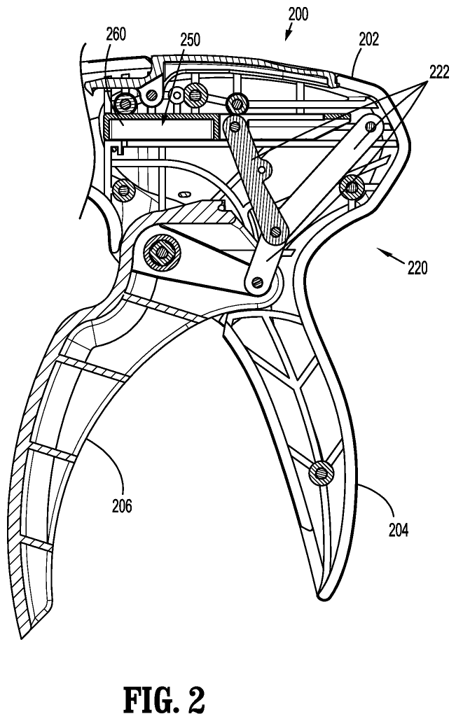

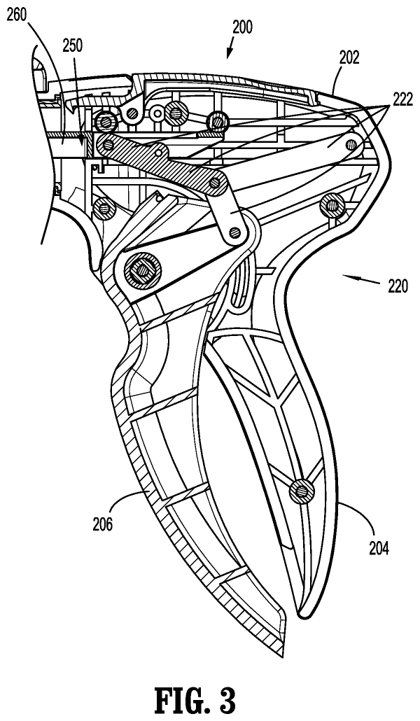

With initial reference to FIG. 1, the surgical clip applier 100 includes a handle assembly 200, an endoscopic or elongated portion 300 extending distally from the handle assembly 200 and defining a longitudinal axis “A-A,” and an end effector 400 disposed adjacent a distal end of the elongated portion 300. A two-part fastener 1000 is supported by the end effector 400. Additionally, an actuation assembly 220 (FIGS. 2-3) and a drive assembly 250 (FIGS. 2-3) are disposed in mechanical cooperation with the handle assembly 200 and are configured to distally advance one part of the two-part fastener 1000.

While details of a particular handle assembly 200, a particular actuation assembly 220, and a particular drive assembly 250 are discussed below, the disclosed surgical clip applier 100 is usable with other types of handle assemblies, actuation assemblies, and drive assemblies without departing from the scope of the disclosure.

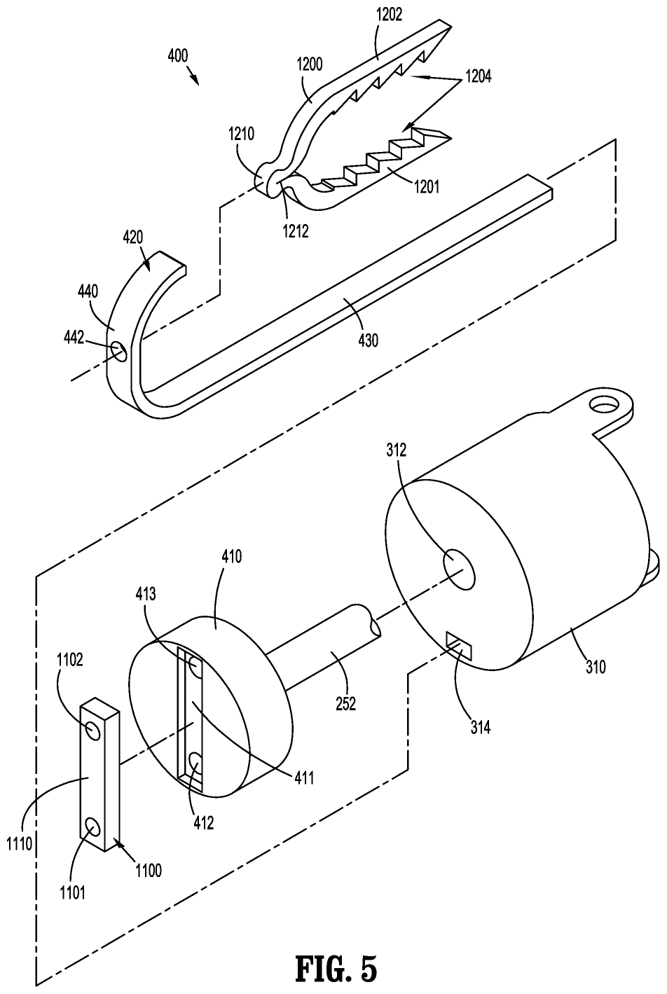

Referring now to FIGS. 1-5, further details of the handle assembly 200, the actuation assembly 220, the drive assembly 250, and the distal portion of the surgical clip applier 100 are shown. The handle assembly 200 includes a handle housing 202, a stationary handle 204, and a pivotable handle 206. The actuation assembly 220 includes a plurality of linkages 222 disposed in operative engagement with the pivotable handle 206. The drive assembly 250 includes a drive shaft 252 (FIG. 5). Generally, actuation of the pivotable handle 206 causes the drive shaft 252 to move distally, which in turn, causes formation of the two-part fastener 1000. Further details of suitable drive assemblies and actuation assemblies are described in pending U.S. patent application Ser. No. 17/572,669, filed on Jan. 11, 2022, and U.S. Pat. No. 11,246,601, the entire contents of each of which are incorporated by reference herein.

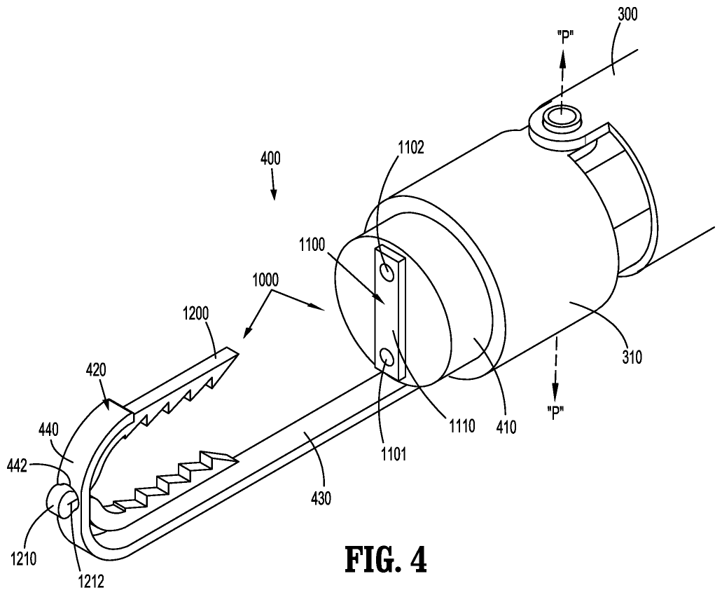

With particular reference to FIGS. 4 and 5, the end effector 400 includes a collar 410 and a distal housing 420, and the two-part fastener 1000 includes a first part or a base 1100 and a second part or a clip 1200. The collar 410 of the end effector 400 supports the base 1100, and the distal housing 420 of the end effector 400 supports the clip 1200.

With continued reference to FIG. 4, the collar 410 of the end effector 400 defines a recess 411, a first aperture or first leg aperture 412, and a second aperture or second leg aperture 413. The recess 411 is configured to releasably receive the base 1100 of the two-part fastener 1000 at least partially therein, e.g., in a press-fit manner. The first aperture 412 and the second aperture 413 are each configured to align with a respective first aperture 1101 and a second aperture 1102 of the base 1100 of the two-part fastener 1000 when the base 1100 is engaged with the recess 411. The first aperture 412 of the collar 410 is configured to allow at least a portion of a first leg 1201 of the clip 1200 to pass through the first aperture 412, and the second aperture 413 of the collar 410 is configured to allow at least a portion of a second leg 1202 of the clip 1200 to pass through the second aperture 413.

Referring now to FIGS. 4-7, the distal housing 420 of the end effector 400 generally includes a leg 430, and a C-shaped portion 440 extending from a distal end of the leg 430. A distal-most end of the C-shaped portion 440 defines an aperture 442 extending therethrough. The aperture 442 is configured to allow a distal tip 1210 of the clip 1200 of the two-part fastener 1000 to extend therethrough. The C-shaped portion 440 of the distal housing 420 also defines a slot 446 extending from the aperture 442 to a cantilevered end 450 of the distal housing 420. The slot 446 is configured to support a portion of the second leg 1202 of the clip 1200 to help stabilize the clip 1200 relative to the distal housing 420, for instance.

With reference to FIGS. 4 and 5, a pivotable member 310 is disposed at a distal end of the elongated portion 300. The pivotable member 310 is pivotable relative to an intermediate portion 305 (FIG. 1) of the elongated portion 300 about axis “P-P” in FIG. 4 to allow for articulation of the end effector 400 relative to the intermediate portion 305 of the elongated portion 300. Pivotable member 310 includes a first aperture 312 for slidingly engaging the drive shaft 252, and a second aperture 314 for fixedly engaging a proximal portion of the leg 430 of the distal housing 420 of the end effector 400.

With continued reference to FIGS. 4 and 5, a distal end of the drive shaft 252 is mechanically coupled to the collar 410, such that distal translation of the drive shaft 252 relative to the handle assembly 200 results in a corresponding distal translation of the collar 410 relative to the distal housing 420 of the end effector 400. As discussed in further detail below, sufficient distal translation of the collar 410 relative to the distal housing 420 causes the base 1100 of the two-part fastener 1000 to engage the clip 1200 of the two-part fastener 1000.

With particular reference to FIGS. 4-7, further details of the two-part fastener 1000 are described. The base 1100 of the two-part fastener 1000 is configured to engage the clip 1200 of the two-part fastener 1000 in a plurality of discrete positions. In each position of engagement, a distance “d” between the base 1100 and the distal tip 1210 of the clip 1200 is different (see FIG. 13, for instance).

The base 1100 may include a generally rectangular body portion 1110 as shown, or body portion 1110 may be of any other suitable shape. The first aperture 1101 and the second aperture 1102 extend through the body portion 1110. The clip 1200 is generally U-shaped and includes the first leg 1201, and the second leg 1202, with the distal tip 1210 interconnecting the first leg 1201 and the second leg 1202.

Each of the first leg 1201 and the second leg 1202 includes a plurality of teeth 1204 for engaging the base 1100 in a ratcheting manner. The plurality of teeth 1204 may be defined by other shapes and other angles than those shown herein without departing from the scope of the present disclosure. Additionally, the amount or number of teeth included in the plurality of teeth 1204 may be more or fewer than the amount shown in the accompanying figures without departing from the scope of the present disclosure.

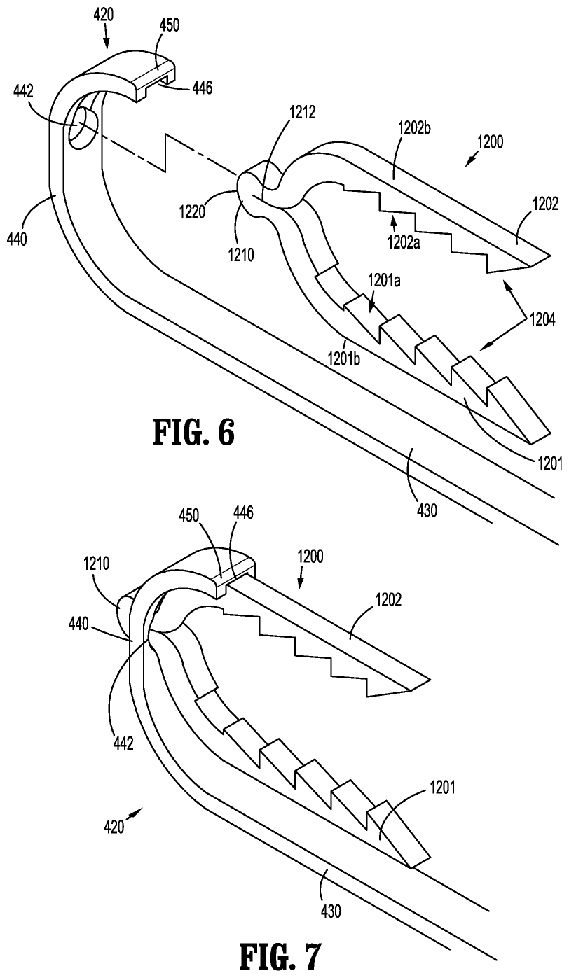

In the illustrated embodiment, an inner-facing surface 1201a of the first leg 1201 and an inner-facing surface 1202a of the second leg 1202 include the plurality of teeth 1204 (see FIG. 6). However, it is also envisioned and within the scope of this disclosure that an outer-facing surface 1201b of the first leg 1201 and/or an outer-facing surface 1202b of the second leg 1202 includes the plurality of teeth 1204.

As shown in FIGS. 4-6, for example, the distal tip 1210 of the clip 1200 protrudes distally from distal ends of the first leg 1201 and the second leg 1202, and generally extends parallel to the longitudinal axis “A-A.” As noted above, at least a portion of the distal tip 1210 is configured to extend through the aperture 442 defined within the C-shaped portion 440 of the distal housing 420 of the end effector 400. Additionally, a slit 1212 extends longitudinally through a portion of the distal tip 1210. As shown in FIG. 4, the slit 1212 extends distally beyond the distal housing 420 of the end effector 400 when the clip 1200 is engaged with the distal housing 420. Further, the slit 1212 does not extend to a distal-most end 1220 of the clip 1200 (FIG. 6). In use, as discussed in further detail below, when a user wants to remove the two-part fastener 1000 from securement on tissue, for instance, the user can cut the distal tip 1210 of the clip 1200 (e.g., from a location at or adjacent the distal-most end 1220 of the clip 1200 to the slit 1212) to separate the first leg 1201 of the clip 1200 from the second leg 1202 of the clip 1200, thereby facilitating removal of the two-part fastener 1000 from tissue.

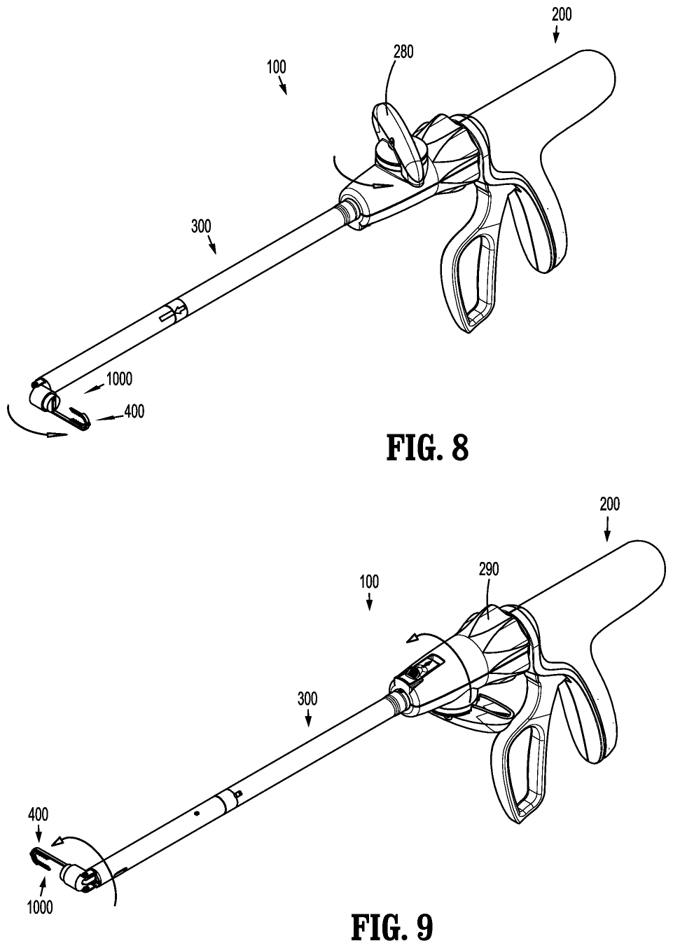

Referring now to FIGS. 8-19, when a user desires to place the two-part fastener 1000 onto a vessel or tissue “T” and/or a catheter “C,” for instance, (e.g., to variably occlude a vessel, or to retain a catheter in a vessel without occluding the vessel such as during cholangiograms), the user initially engages the base 1100 of the two-part fastener 1000 with the collar 410 of the end effector 400, and engages the clip 1200 of the two-part fastener 1000 with the distal housing 420 of the end effector 400. Alternatively, the user ensures the two-part fastener 1000 is properly engaged with the end effector 400. Next, the user can position the end effector 400 at a desired location by articulating the end effector 400 by actuating an articulation knob 280 (FIG. 8) and/or by rotating the end effector 400 by actuating a rotation knob 290 (FIG. 9), for instance.

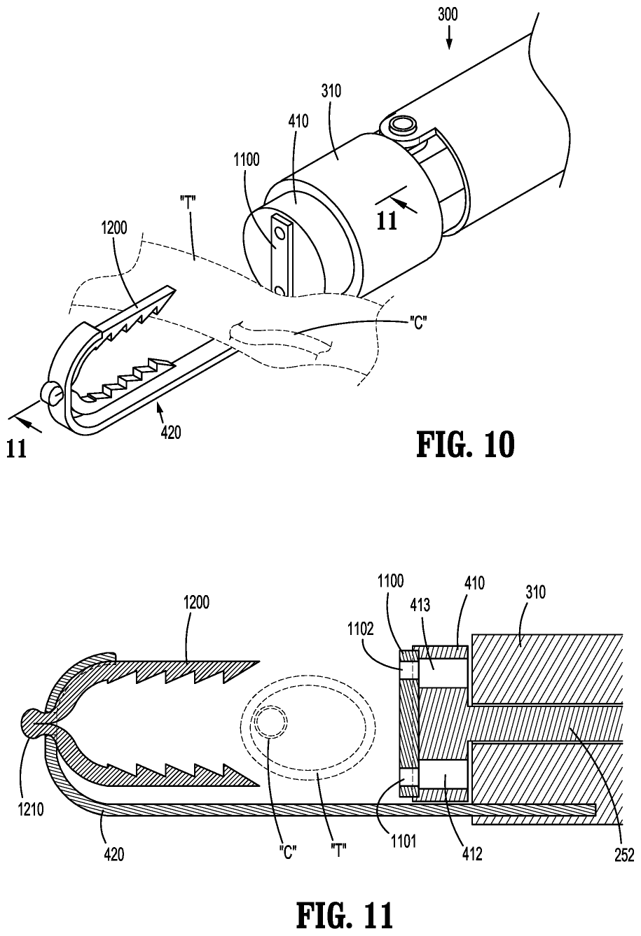

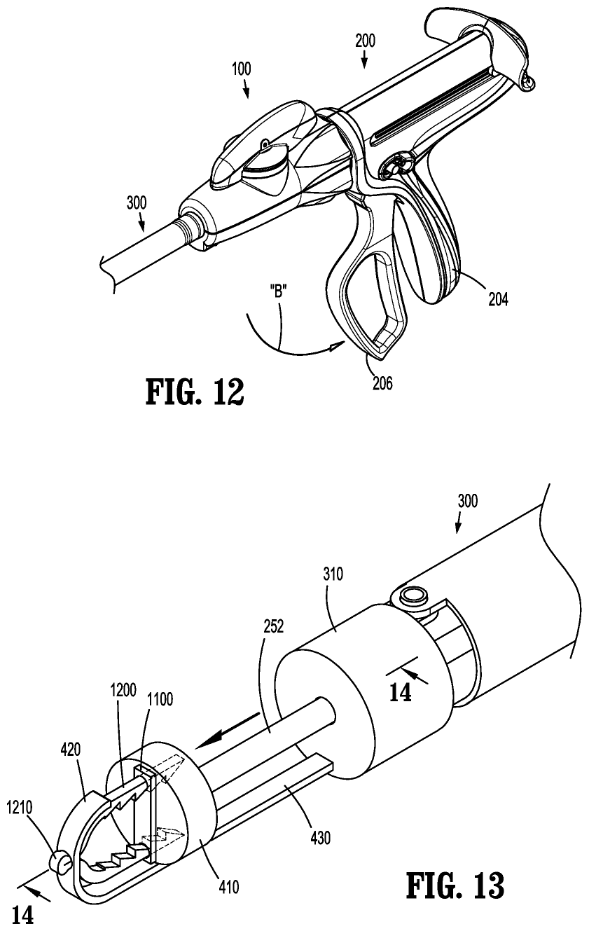

After the end effector 400 is in its desired position (e.g., FIG. 10), the collar 410 of the end effector 400 is moved distally relative to the distal housing 420 of the end effector 400 by actuating the pivotable handle 206 in the general direction of arrow “B” (FIG. 12), or another actuation mechanism, thereby advancing the drive shaft 252 distally, which moves the base 1100 toward the clip 1200 of the two-part fastener 1000. (Other structures for moving the collar 410 relative to the distal housing 420, such as depressing a button, are also envisioned.) When the base 1100 moves a sufficient distance relative to the clip 1200, the first aperture 1101 of the base 1100 surrounds or engages a proximal portion of the first leg 1201 of the clip 1200, and the second aperture 1102 of the base 1100 surrounds or engages a proximal portion of the second leg 1202 of the clip 1200.

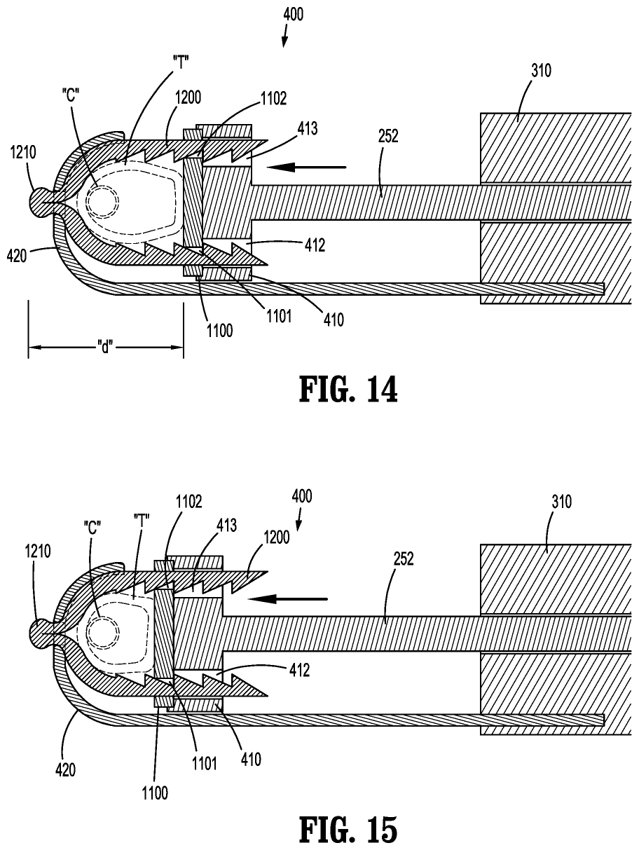

With reference to FIGS. 13-15, the collar 410 of the end effector 400 is moved closer to the distal housing 420 until a desired compression or degree of crimp of the tissue “T” and/or the catheter “C” between the base 1100 and the clip 1200 of the two-part fastener 1000 is achieved. FIG. 14 illustrates the base 1100 in a first discrete position relative to the clip 1200, and FIG. 15 illustrates the base 1100 in a second discrete position relative to the clip 1200. There are several discrete positions of the base 1100 relative to the clip 1200 that allow the catheter “C” to be fixed to the tissue “T” without occluding the vessel.

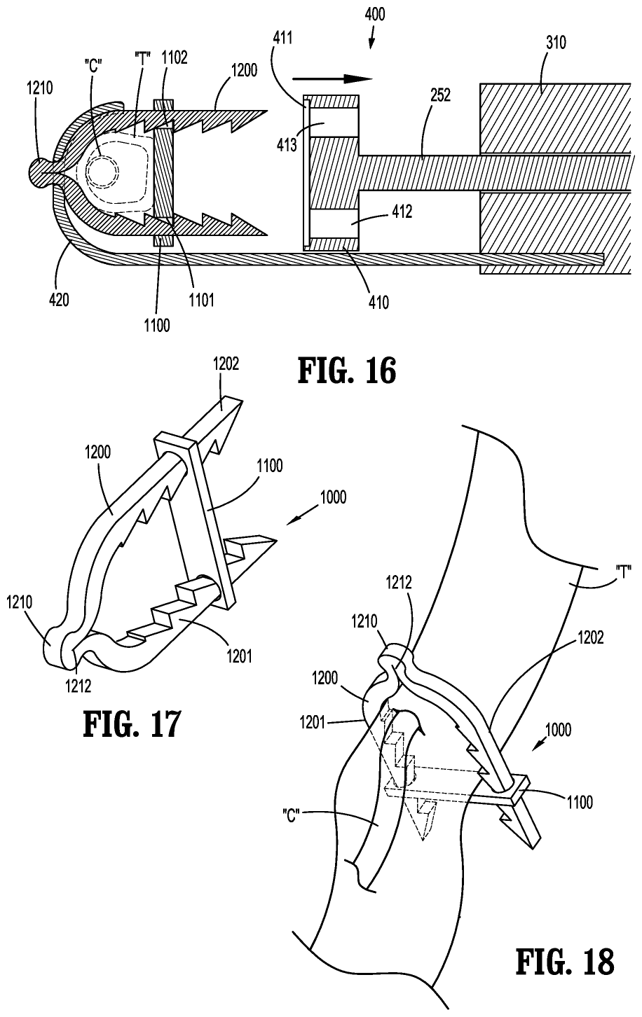

Referring now to FIG. 16, after the two-part fastener 1000 is desirably placed on the tissue “T” and/or the catheter “C,” the drive shaft 252 is moved proximally (e.g., by moving the pivotable handle 206 away from the stationary handle 204), thereby retracting the collar 410 of the end effector 400 relative to the distal housing 420. As shown, the base 1100 remains engaged with the clip 1200 of the two-part fastener 1000. Additionally, moving the surgical clip applier 100 distally relative to the tissue “T” causes the distal housing 420 of the end effector 400 to move out of engagement with the distal tip 1210 of the clip 1200 of the two-part fastener 1000, thereby leaving the two-part fastener 1000 secured to the tissue “T” and/or the catheter “C” (FIG. 18).

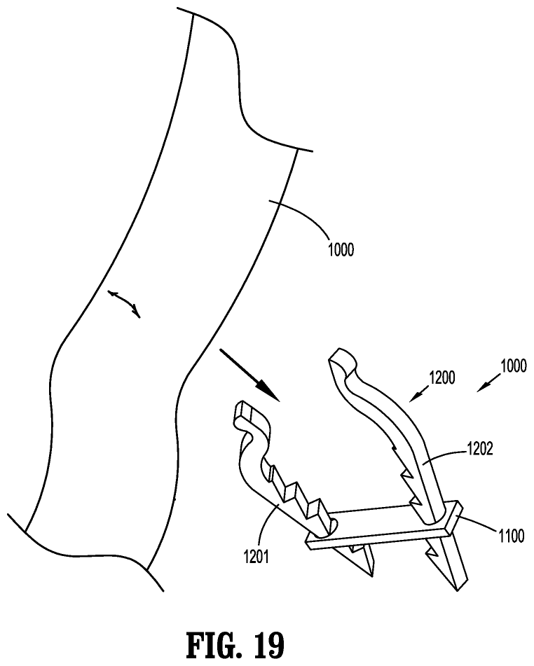

After the surgical clip applier 100 is removed, a surgical procedure, such as a cholangiogram, may be performed. When the surgical procedure is complete, for instance, the two-part fastener 1000 may be removed from the tissue “T” and/or the catheter “C.” To remove the two-part fastener 1000, the user may cut or sufficiently clamp the distal tip 1210 of the clip 1200, e.g., adjacent the slit 1212, to separate the first leg 1201 of the clip 1200 from the second leg 1202 of the clip 1200 (see FIG. 19). After the first leg 1201 of the clip 1200 and the second leg 1202 of the clip 1200 have been separated, the two-part fastener 1000 can easily be removed from a body cavity, for instance (e.g., using graspers).

The present disclosure also includes a surgical clip-applying system, which includes both the surgical clip applier 100 and the two-part fastener 1000. Additionally, the present disclosure includes methods of installing and/or removing the two-part fastener 1000, and methods of fixing a catheter to a vessel using the surgical clip applier 100 and the two-part fastener 1000.

The various embodiments disclosed herein may also be configured to work with robotic surgical systems and what is commonly referred to as “Telesurgery.” Such systems employ various robotic elements to assist the surgeon in the operating theater and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include, remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prepare the patient for surgery and configure the robotic surgical system with one or more of the instruments disclosed herein while another surgeon (or group of surgeons) remotely control the instruments via the robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the surgeon to produce a corresponding movement of the working ends of any type of surgical instrument (e.g., end effectors, graspers, knifes, scissors, etc.) which may complement the use of one or more of the embodiments described herein. The movement of the master handles may be scaled so that the working ends have a corresponding movement that is different, smaller or larger, than the movement performed by the operating hands of the surgeon. The scale factor or gearing ratio may be adjustable so that the operator can control the resolution of the working ends of the surgical instrument(s).

The master handles may include various sensors to provide feedback to the surgeon relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the surgeon with enhanced tactile feedback simulating actual operating conditions. The master handles may also include a variety of different actuators for delicate tissue manipulation or treatment further enhancing the surgeon's ability to mimic actual operating conditions.

With reference to FIG. 20, a surgical system, such as, for example, a robotic surgical system is shown generally as surgical system 2000 and is usable with the surgical clip applier 100, or portions thereof, of the disclosure. Surgical system 2000 generally includes a plurality of robotic arms 2002, 2003, a control device 2004, and an operating console 2005 coupled with control device 2004. Operating console 2005 includes a display device 2006, which is set up in particular to display three-dimensional images; and manual input devices 2007, 2008, by means of which a person (not shown), for example a surgeon, is able to telemanipulate robotic arms 2002, 2003 in a first operating mode, as known in principle to a person skilled in the art.

Each of the robotic arms 2002, 2003 is composed of a plurality of members, which are connected through joints. System 2000 also includes an instrument drive unit 2200 connected to distal ends of each of robotic arms 2002, 2003. The surgical clip applier 100, or portions thereof, may be attached to the instrument drive unit 2200, in accordance with any one of several embodiments disclosed herein, as will be described in greater detail below.

Robotic arms 2002, 2003 may be driven by electric drives (not shown) that are connected to control device 2004. Control device 2004 (e.g., a computer) is set up to activate the drives, in particular by means of a computer program, in such a way that robotic arms 2002, 2003, their instrument drive units 2200 and thus the surgical clip applier 100 (including the end-effector 400) execute a desired movement according to a movement defined by means of manual input devices 2007, 2008. Control device 2004 may also be set up in such a way that it regulates the movement of robotic arms 2002, 2003 and/or of the drives.

Surgical system 2000 is configured for use on a patient 2013 lying on a patient table 2012 to be treated in a minimally invasive manner by means of the surgical clip applier 100. Surgical system 2000 may also include more than two robotic arms 2002, 2003, the additional robotic arms likewise being connected to control device 2004 and being telemanipulatable by means of operating console 2005.

Reference may be made to U.S. Pat. No. 8,828,023, entitled “Medical Workstation,” the entire content of which is incorporated herein by reference, for a detailed discussion of the construction and operation of surgical system 2000.

It should be understood that the foregoing description is only illustrative of the disclosure. Various alternatives and modifications can be devised by those skilled in the art without departing from the disclosure. Accordingly, this disclosure is intended to embrace all such alternatives, modifications and variances. The embodiments described with reference to the attached drawing figures are presented only to demonstrate certain examples of the disclosure. Other elements, steps, methods and techniques that are insubstantially different from those described above and/or in the appended claims are also intended to be within the scope of the disclosure.

What is claimed is:

1. A surgical clip applier, comprising:
an elongated portion defining a longitudinal axis and including a distal end;
a drive shaft extending at least partially through the elongated portion; and
an end effector disposed adjacent the distal end of the elongated portion, the end effector including a collar and a distal housing, the collar coupled to the drive shaft and movable relative to the distal housing, the collar configured to support a base of a two-part fastener, the distal housing including a leg and a C-shaped portion extending from a distal end of the leg, the C-shaped portion of the distal housing configured to support a clip of the two-part fastener.

2. The surgical clip applier according to claim 1, wherein a distal-most end of the C-shaped portion of the distal housing defines an aperture.

3. The surgical clip applier according to claim 2, wherein the C-shaped portion of the distal housing defines a slot extending laterally from the aperture, the slot configured to support a portion of the clip of the two-part fastener.

4. The surgical clip applier according to claim 1, wherein the distal housing is fixed from longitudinal movement relative to the elongated portion.

5. The surgical clip applier according to claim 1, wherein the collar of the end effector defines a recess for accepting the base of the two-part fastener partially in the recess.

6. The surgical clip applier according to claim 1, wherein the collar of the end effector defines a first leg aperture and a second leg aperture, the first leg aperture configured to allow a first leg of the clip of the two-part fastener to extend through the collar, the second leg aperture configured to allow a second leg of the clip of the two-part fastener to extend through the collar.

7. The surgical clip applier according to claim 5, wherein the collar of the end effector defines a first leg aperture and a second leg aperture, the first leg aperture defined within the recess of the collar and configured to allow a first leg of the clip of the two-part fastener to extend through the collar, the second leg aperture defined within the recess of the collar and configured to allow a second leg of the clip of the two-part fastener to extend through the collar.

8. A surgical clip-applying system, comprising:
a surgical clip applier, including:
an elongated portion defining a longitudinal axis and including a distal end; and
an end effector disposed adjacent the distal end of the elongated portion, the end effector including a collar and a distal housing, at least one of the collar or the distal housing being movable along the longitudinal axis relative to the elongated portion; and
a two-part fastener, including:
a base configured to be supported by the collar of the end effector of the surgical clip applier; and
a clip configured to be supported by the distal housing of the end effector of the surgical clip applier, and including a first leg and a second leg,
wherein a predetermined amount movement of at least one of the collar or the distal housing relative to the elongated portion causes the base of the two-part fastener to engage the clip of the two-part fastener.

9. The surgical clip-applying system according to claim 8, wherein the distal housing of the end effector includes a C-shaped portion.

10. The surgical clip-applying system according to claim 8, wherein the base of the two-part fastener defines a first aperture and a second aperture.

11. The surgical clip-applying system according to claim 10, wherein the clip of the two-part fastener is C-shaped and includes a first leg and a second leg, the first leg configured to selectively extend through the first aperture of the base of the two-part fastener, and the second leg configured to selectively extend through the second aperture of the base of the two-part fastener.

12. The surgical clip-applying system according to claim 11, wherein each of the first leg and the second leg of the clip of the two-part fastener includes a plurality of teeth configured to engage the base of the two-part fastener in a ratcheting manner.

13. The surgical clip-applying system according to claim 8, wherein a distal-most end of the distal housing of the end effector defines an aperture.

14. The surgical clip-applying system according to claim 13, wherein a distal end of the clip of the two-part fastener includes a distal tip configured to selectively extend through the aperture of the distal housing of the end effector.

15. The surgical clip-applying system according to claim 14, wherein the distal tip of the clip of the two-part fastener defines a slit extending generally parallel to the first leg of the clip of the two-part fastener.

16. The surgical clip-applying system according to claim 15, wherein when the distal tip of the clip of the two-part fastener extends through the aperture of the distal housing of the end effector, and the slit of the distal tip extends distally beyond the distal-most end of the distal housing of the end effector.

17. The surgical clip-applying system according to claim 8, wherein the base of the two-part fastener is supported partially within a recess of the collar of the end effector.

18. The surgical clip-applying system according to claim 8, wherein the collar of the end effector defines a first leg aperture and a second leg aperture, and the base of the two-part fastener defines a first leg aperture and a second leg aperture.

19. The surgical clip-applying system according to claim 18, wherein after a predetermined amount of movement of the at least one of the collar or the distal housing relative to the elongated portion, the first leg of the clip of the two-part fastener extends through the first leg aperture of the collar and through the first leg aperture of the base of the two-part fastener, and the second leg of the clip of the two-part fastener extends through the second leg aperture of the collar and through the second leg aperture of the base of the two-part fastener.
